(19) United States
(12) Patent Application Publication
(10) Pub. No.: US 2026/0340391 A1
(43) Pub. Date: Oct. 29, 2026

(54) CHAINED CLASSIFICATION BY COMPONENTS FOR INTERPRETABLE MACHINE LEARNING

(71) Applicant: NEC Laboratories Europe GmbH, Heidelberg (DE)

(72) Inventors: Sascha SARALAJEW, Heidelberg (DE); Carolin LAWRENCE, Heidelberg (DE)

(21) Appl. No.: 19/107,046

(22) PCT Filed: Apr. 28, 2023
(86) PCT No.: PCT/EP2023/061315

§ 371 Date: Feb. 27, 2025

(30) Foreign Application Priority Data
Oct. 21, 2022 (EP) 22203003

(51) Int. Cl. G06N 3/084; G06N 3/047

(52) U.S. Cl. CPC G06N 3/084; G06N 3/047

(57) ABSTRACT

A computer-implemented method for creating an interpretable machine learning classification includes defining the model by setting up a chained classification-by-components (CBC) architecture including a number of chained CBC blocks. The model is end-to-end trained on available input data including forward-propagating samples of input data through the chained CBC architecture. An output of each CBC block of the chained CBC architecture is interpreted, except a last CBC block, as a possibility vector of concepts detected in a respective one of the CBC blocks.

### CROSS REFERENCE TO RELATED APPLICATIONS

This application is a U.S. National Phase application under 35 U.S.C. § 371 of International Application No. PCT/EP2023/061315, filed on Apr. 28, 2023, and claims benefit to European Patent Application No. 22203003.3, filed on Oct. 21, 2022. The International Application was published in English on Apr. 25, 2024 as WO 2024/083360 A1 under PCT Article 21(2).

### FIELD

The present disclosure relates to computer-implemented methods and systems for creating an interpretable machine learning classification model.

### BACKGROUND

Embodiments of the present disclosure address the problem of learning fully interpretable deep probabilistic classification models. Currently existing interpretable models, like k-nearest neighbour classifiers or linear regression models, are shallow so that they are limited in capacity, leading to the fact that the complexity of problems they can solve efficiently is limited.

To overcome this limitation, deployed classification models are often deep neural networks (DNNs). This, however, comes at the cost of deploying uninterpretable models, since due to their design, considered as black boxes, it is in general hard to gain insights into their decision making process. This leads to problems in situations where there is an interest in understanding the classification decision like in high-stakes areas such as, e.g., the medical domain. Thus, in summary, current machine learning models have the problem that they are either interpretable but limited in capacity (shallow models), or non-interpretable but unlimited in capacity (deep models).

### SUMMARY

In an embodiment, the present disclosure provides a computer-implemented method for creating an interpretable machine learning classification model, the method being implemented in one or more processors connected to a memory. The method defines the model by setting up a chained classification-by-components (CBC) architecture including a number of chained CBC blocks. The model is end-to-end trained on available input data including forward-propagating samples of input data through the chained CBC architecture. An output of each CBC block of the chained CBC architecture is interpreted, except a last CBC block, as a possibility vector of concepts detected in a respective one of the CBC blocks.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view illustrating an ordinary CBC structure according to prior art;

FIG. 2 is a schematic view illustrating an example realization of a classification process of a CBC architecture on a digit classification task, according to an embodiment of the present disclosure;

FIG. 3 is a schematic view illustrating a system of a chained CBC network, according to an embodiment of the present disclosure; and

FIG. 4 is a schematic view illustrating a method for creating a fully interpretable machine learning method, according to an embodiment of the present disclosure.

### DETAILED DESCRIPTION

In accordance with an embodiment, the present disclosure improves and further develops a method and a system of the kind described above in such a way that deep neural network architectures are provided that are fully interpretable.

In accordance another embodiment, the present disclosure provides a computer-implemented method for creating an interpretable machine learning classification model, the method being implemented in one or more processors connected to a memory, the method comprising: defining the model by setting up a chained classification-by-components, CBC, architecture including a number of chained CBC blocks; end-to-end training of the model on available input data including forward-propagating samples of input data through the chained CBC architecture; and interpreting the output of each CBC block of the chained CBC architecture, except the last one, as a possibility vector of concepts detected in the respective one of the CBC blocks.

According to the disclosure it has first been recognized that the aforementioned problem can be solved by presenting an architecture that can be deep but still interpretable by chaining classification-by-components networks (chained CBCs). According to embodiments, on the heart of this architecture is the CBC block that may be directly related to a probability tree diagram so that each parameter in the block has probabilistic interpretation. According to a further embodiment, by interpreting the output of a CBC block as probability values for detected primitives and by applying binary gates, these CBC blocks can be chained such that the intermediate weights in the network are associated with the probabilistic interpretation, which makes the reasoning process of the network understandable. As a result, the model capacity is increased and the interpretability is preserved at the same time.

Embodiments of the present disclosure provide a deep machine learning architecture for classification that is constraint to be fully interpretable. In particular, each parameter in the architecture is associated with a probabilistic meaning so that the internal reasoning process of the model can be understood by experts. This makes the proposed architecture superior if machine learning methods have to be applied for high-stakes decisions.

According to embodiments, the CBC blocks have a two-stage approach, wherein in the first stage, the input is analysed, and matching is performed with respect to a probability measure. In the second stage, the probabilities of the detected components are analysed to find the reason for the class allocation. The reasoning is done by computing the probability of detection for each class.

According to an embodiment, each of the CBC blocks may include two stages. The first stage may be configured to determine, for each input, detection probability measures based on an analysis of the respective inputs for matches with a given set of primitives. The second stage may be configured to analyse the probabilities of the detected primitives of the set of primitives to reason about the present concepts in the given input.

According to an embodiment, a probabilistic nature of the chained CBC architecture may be ensured by applying a binarization technique in between subsequent CBC block that is configured to binarize the possibility vectors. Alternatively or additionally, it may be provided that the probabilistic nature of the chained CBC architecture is ensured by applying a binarization technique on all detected primitives after the first CBC block of the chained CBC network. For instance, the binarization technique may apply a Gumble softmax concept or the Implicit Maximum Likelihood Estimation (I-MLE) framework.

According to an embodiment, it may be provided that the model is updated based on a back-propagation of classification errors.

According to an embodiment, it may be provided that the trained model is used to predict the classes of new data samples and to interpret the classification process by sample-based local explanations and/or model-based global explanations.

For instance, according to embodiment, the model may be trained on health related patient data, including at least one of blood pressure, temperature and EEG, and/or image data from the patients. The trained model may then be used to predict one or more classified diseases of a patient and to obtain explanations of the intermediate decision process of the model for verification by a medical expert.

According to another embodiment, the model may be trained on blood markers from blood test results of patients including diagnosed diseases. After training, the model's intermediate representations may be inspected for generating knowledge about yet unknown correlations between blood markers and diseases.

According to yet another embodiment, the model may be trained on satellite image data of the earth. After training, the trained model may then be used to predict unknown indicators and their relations for certain nature losses and to obtain knowledge from the model's intermediate representations regarding which passed policies and/or measures were responsible for a given nature loss.

This disclosure can be used for several anticipated medical/healthcare use cases, for example, transparent disease classification and knowledge discovery for disease classification by analysing markers of blood tests.

There are several ways how to design and further develop the teaching of the present disclosure in an advantageous way. To this end, it is to be referred to the dependent claims on the one hand and to the following explanation of preferred embodiments of the disclosure by way of example, illustrated by the figure on the other hand. In connection with the explanation of the preferred embodiments of the disclosure by the aid of the figure, generally preferred embodiments and further developments of the teaching will be explained. In the drawing

Currently, there are two main themes to tackle the interpretability of machine learning methods. The first one is to build post-hoc explanations. These explanations are often based on approximations or relaxations of the network after the network is trained. The problem with these explanations is that it is unclear to which extent they are faithful (i.e., reflect the true reasoning process of the model). Methods that derive the explanations as part of the training process can also be considered in this category.

The second approach are models that have a built-in interpretability, like k-nearest neighbours methods. Built-in interpretability means that by design/through constraints (not by adding regularization terms to the training loss), each parameter or subset of parameters has a clear meaning in the classification process that can be understood by a human expert. Moreover, this also means that the interpretation of these parameters reflects the true reasoning process of the model. Such models that are interpretable by design are preferred for high-stakes decisions. However, currently, there is no method available that can build deep fully interpretable architectures so that complex classification problems can be addressed.

Embodiments of the present disclosure address this problem by presenting an approach to create deep fully interpretable architectures that are end-to-end trainable.

For example, a deep machine learning model, e.g. for classification learning, is fully interpretable, if each parameter (or set of parameters) in the model is associated to a probability value of an event like, e.g., “the probability that high blood pressure and fatigue must be observed for diabetes.” Embodiments of the present disclosure provide such a model for different kind of data inputs for supervised learning based on a clear probabilistic structure. In an embodiment, the model uses the probabilistic structure of a Classification-by-Components (CBC) network, as disclosed in Saralajew et al.: “Classification-by-Components: Probabilistic Modeling of Reasoning over a Set of Components”, in NeurIPS 2019, which is hereby incorporated herein in its entirety by way of reference. CBC was introduced as standalone network for classification consisting of two layers and predefined/learned components.

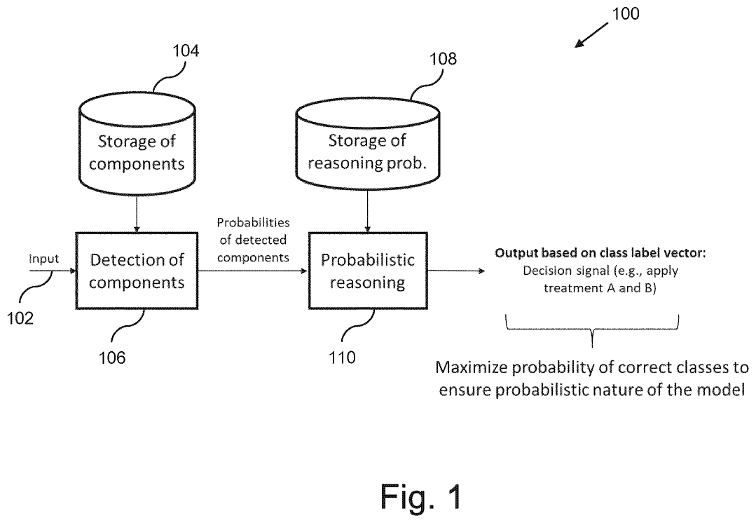

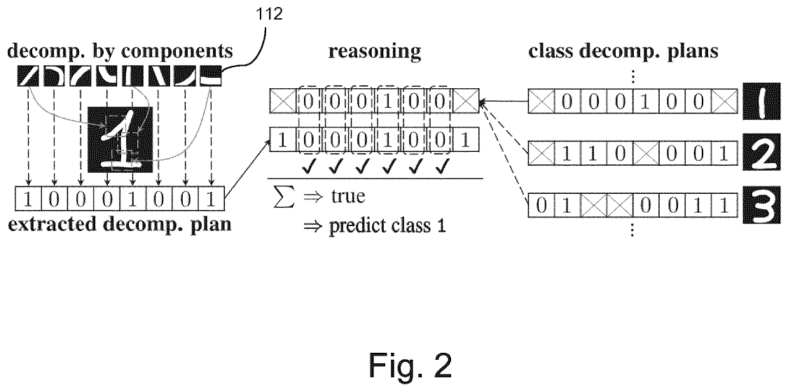

FIG. 1 provides an overview of an ordinary CBC structure 100 according to prior art, while FIG. 2 illustrates an example realization of a classification process of a CBC architecture on a digit classification task.

The idea of CBCs is that the classification of inputs 102 follows a two-stage approach. At a first stage, the input 102 is analysed by using, from a storage 104, a set of components 112 (learned or predefined). This analysis is done by a detection element 106 searching for matches with the components 112 with respect to an appropriate probability measure (see first part in FIG. 1 and left part in FIG. 2). A corresponding detection probability measure for a given component 112 is high if the component 112 has a match with the input 102 (or parts of) and becomes small otherwise.

After this first stage, in a second stage, which includes a storage 108 of reasoning probabilities, the probabilities of the detected components 112 are analysed to reason about the class of the given input 102 (see second part in FIG. 1 and right and middle part in FIG. 2). This reasoning is done by computing, by probabilistic reasoning element 110, for each class the probability that the respective components 112 must be detected and have been detected in the input (illustrated by matching “1” in FIG. 2) or must not be detected and have not been detected in the input (illustrated by matching “0” in FIG. 2).

Additionally, to avoid that each component 112 has to be considered for the computation of a class probability, the model can also learn that a component 112 is ignored for the decision process (illustrated by the “x” in FIG. 2). Overall, the probability for a given class c can be computed by (cf. formula (1) on page 3 of the Saralajew et al. paper referenced above):

$${p}_{c} ( x ) = \frac{{( d ( x ) )}^{T} \cdot {r}_{c}^{+} + {( 1 - d ( x ) )}^{T} \cdot {r}_{c}^{-}}{{1}^{T} \cdot ( {r}_{c}^{+} + {r}_{c}^{-} )}$$

where d(x) models the detection probabilities for the components 112 and

$${r}_{c}^{\pm }$$

models the reasoning process.
A special feature about this model is that it is related to a probability tree diagram and, therefore, the entire model follows a probabilistic model and is fully interpretable. Additionally, the model is fully differentiable. Consequently, all model parameters (i.e. components 112 and the reasoning probabilities) can be learned via end-to-end training by maximizing the correct class probability. To emphasize, due to the relation to a probability tree diagram, each parameter in the model can be interpreted and, thus, has a precise meaning. This property can be used to interpret, for instance, why a model is fooled by an adversarial example or what caused the model to predict a certain class.
As an example, for the computation of the detection probability dₖ(x) for component κₖ, it may be provided to consider the rbf (radial basis function)-kernel:

$${d}_{k} ( x ) = exp ( - \frac{{d}_{E}^{2} ( x , {κ}_{k} )}{σ} )$$

where d_{E} is the Euclidean distance and σ is a temperature parameter.

Even if the model has a superior interpretability compared to standard neural networks, it is limited to the two-stage (two-layer) architecture described above in connection with FIGS. 1 and 2. It is not possible to simply chain these CBC blocks, i.e. to perform a successive application of these two-layers, because after chaining, the probabilistic nature is no longer given as it can be necessary that the output of each “probabilistic reasoning” block is optimized towards a vector {0,1}^{C} (also illustrated in FIG. 1). Finally, it should be noted that all these operations can be extended to sliding operations such that a convolutional-like architecture can be build.

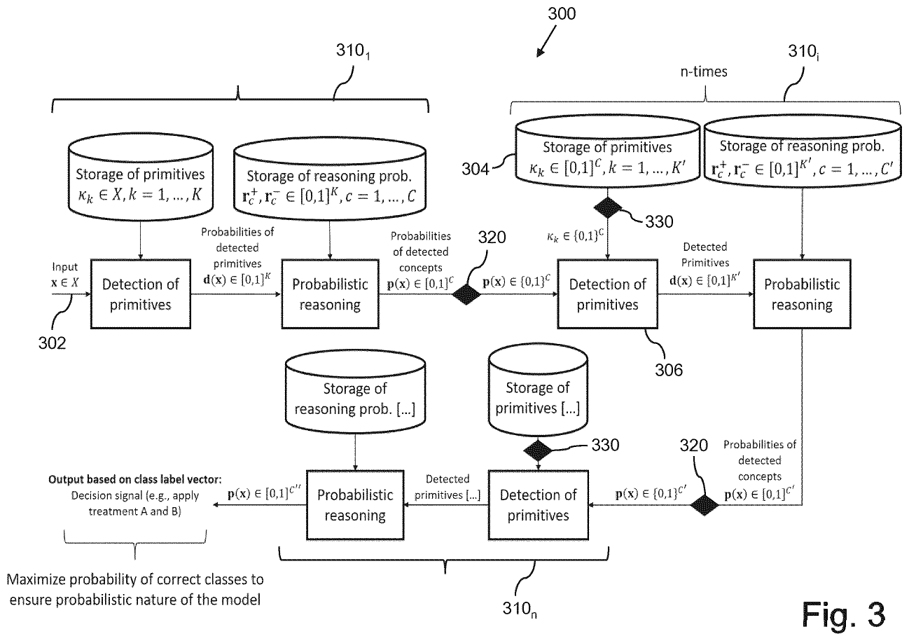

Embodiments of the present disclosure provide systems and methods that overcome this limitation and present an approach to chain CBC networks so that higher-level components and reasoning concepts can be learned. In a sense, this could be compared to neural networks that learn in deeper layers more complex, higher-level features. The overall concept of a chained CBC network 300 according to an embodiment of the present disclosure is depicted in FIG. 3, where the network 300 includes n CBC blocks 310 (three of them being explicitly shown).

First, in contrast to conventional CBCs as described above, embodiments of the present disclosure consider the components in CBCs as “primitives” to clearly differentiate from the “components” used in conventional CBCs. According to embodiments, these primitives are learned during training, and may encode, together with the reasoning process, increasingly complex concepts of the respective domain.

With respect to the terminology used herein, it should be noted that, basically, in traditional CBCs, components are combined to reason about classes such as: “Detected component A, but not detected component B, consequently the input sample belongs to class 1”. In the context of the present disclosure, the situation may be like (for the first CBC block 310₁ of the CBC network 300): “Detected primitives A and B, but not primitive C, consequently the input sample builds concept 1”. In this regard, the term “primitive” is to be understood in the sense of a primitive element (of whatever kind) with a level of complexity lower than the level of complexity of “concepts”. As such, the term “concept” denotes a construct on a higher-level of complexity, which is based on combinations of detected primitives.

Then, in the next CBC block, i.e. CBC block 310₂, of the CBC network 300, the output of detected concepts from the previous CBC block, i.e. “concept 1”, “concept 2”, and so on, from CBC block 310₁ in the above example, may be considered as input to CBC block 310₂. Then, this input is compared with the primitives of CBC block 310₂ so that a template matching over concepts given primitives is performed. The primitives of CBC block 310₂ encode which concepts should be or should not be detected in the input. Based on the detection probabilities of CBC block 310₂, new higher-level concepts are formed by the reasoning process such as “Concept 1 detected and concept 2 not detected, consequently this builds higher-level concept ALPHA”. In the next block, in the simplest cast, the primitives might be concepts ALPHA (and, remaining in the notation, e.g., BETA, GAMMA, etc.). This approach is repeatedly performed from block to block, so that the “concepts” are potentially getting more and more complex.

As an example on an image level, assuming digit recognition, the above mechanism can be thought of as follows: First, primitives may be horizontal, vertical, and diagonal lines (of multiples of 45 degree). Via the first reasoning, these primitives may be combined to crosses, corners, V-shapes, etc., which are the concepts of the first block. Then, in the next CBC block, these concepts are considered as primitives and again matches are trying to be found like the first primitive is a cross overlaid with a V-shape and so on. These detected high-level primitives are then recombined by the next reasoning block.

Accordingly, the method may include detecting, by a CBC block of the CBC network, a certain combination of predefined primitives, and deriving the existence of one or more concepts based on the detected combination of primitives. Further, the method may include using, by a subsequent CBC block of the CBC network, the derived one or more concepts as primitives for further recombination.

Second, in contrast to conventional CBCs and in accordance with embodiments of the present disclosure, the output of intermediate CBC blocks 310ᵢ (i=2, . . . , n−1) of the chained CBC network 300 is interpreted as a possibility vector of the detected concepts. According to an embodiment, to ensure the probabilistic nature of the CBC blocks 310, when the possibility vector is fed into another CBC block 310, the possibility vector or the primitives or both are binarized. For binarization of the possibility vectors, as shown in FIG. 3, binary gates 320 may be implemented between subsequent CBC blocks 310 of the chain 300. Alternatively or additionally, for binarization of the primitives, binary gates 330 may be implemented within each CBC block 310 (except the first one 310₁) between a storage 304 of primitives and a detection element 306 that is configured to search an input 320 for matches with the component's primitives. This means that, according to embodiments of the present disclosure, at least one of the two vectors is made binary, before the computation of the detection probabilities of the primitives is executed.

It has been recognized that without such binary gates 320, the probabilistic property is not preserved (except for the last block 310ₙ of the chain 300) because a high detection probability dₖ(x)=1 can be achieved for any vector, if x=κₖ. This means, that a high probability can be achieved if the possibility vector of the detected concepts equals the primitive vector, for instance, if p(x)=κₖ=(0.5, 0.5, . . . , 0.5). Consequently, this would result in a high detection probability for a primitive even if the probability of detected concepts reflects uncertainty. By the binary gates 320, this issue is resolved so that the force to produce {0,1}-probability-vectors is back-propagated through the network when the output probability is maximized, preserving the probabilistic nature of CBC blocks 310 in the chained version 300. To be precise, with the binary gates 320, a high output probability can only be achieved if all probabilities tend to be {0,1}-vectors.

As shown in FIG. 3, the chained CBC network 300 may produce an output based on class label vectors, which may be interpreted as a decision signal (e.g., to apply either a treatment A or a treatment B).

According to embodiments of the present disclosure, the binarization can be done by applying a Gumble softmax or the Implicit-MLE concept, as disclosed in Niepert et al.: “Implicit MLE: Backpropagating Through Discrete Exponential Family Distributions.” NeurIPS 2021, which is hereby incorporated herein in its entirety by way of reference. The Implicit-MLE concept is beneficial as it allows the integration of additional constraints like sparsity of the {0,1}-vector. In principle, it suffices to apply the binarization on the probabilities of detected concepts or on the primitives. However, the latter one is beneficial as it avoids that estimated gradients over the binary gates are back-propagated through the entire network so that learning instabilities are avoided. Additionally, the binarization of primitives improves the comprehensibility of the learned primitives because only two states per feature are possible.

An important advantage of a chained CBC architecture 300 as disclosed herein compared to traditional deep neural networks is that it does via the chained CBC blocks 310 an efficient feature engineering that is end-to-end trainable and fully interpretable. Thus, each parameter in the network is associated to a clear probabilistic meaning, allowing to interpret the entire feature engineering and classification process.

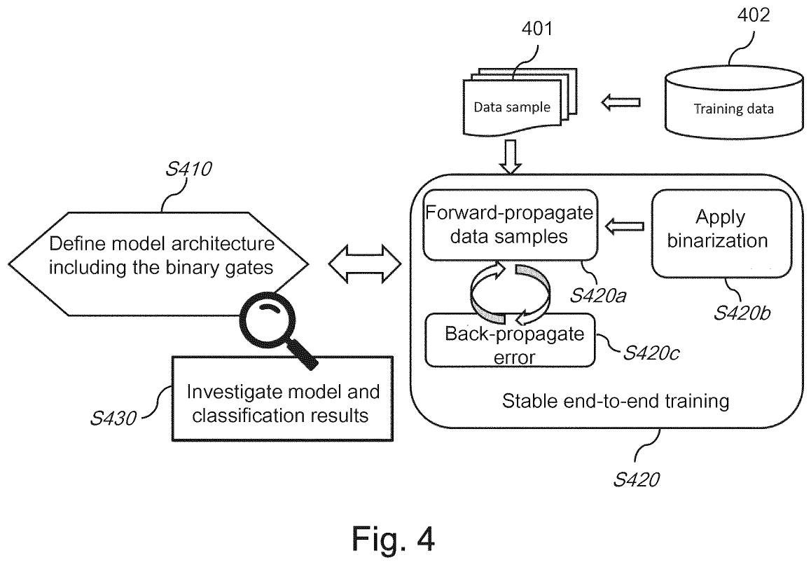

FIG. 4 is a schematic view illustrating a method for creating a fully interpretable machine learning method according to an embodiment of the present disclosure. As shown at S410, the method includes defining an architecture of the model (e.g., an architecture as the CBC chain 300 shown in FIG. 3) by specifying the number of how many CBC blocks and primitives/concepts per CBC block the model should be composed of and by chaining the respective number of CBC blocks accordingly. This step also includes the specification of the binary gates between the CBC blocks and the selection of an appropriate discretization approach to ensure the probabilistic nature of the model (e.g., by either binarizing the primitives or the possibility vectors).

After that and as shown at S420, the method may include a step of performing an end-to-end training of the model on available data, e.g. data samples 401 extracted from a pool of training data 402.

The end-to-end training of the model may include a forward propagation of the data samples 401 through the chained CBC model, as shown at step S420a. During this forward propagation the output of each CBC block of the chained CBC model, except the last one, is interpreted as a possibility vector of detected concept.

As shown at step S420b, in the context of the end-to-end training of the model it may be provided that a binarization technique is applied during the forward computation. This may be performed by binarizing all primitives after the first CBC block of the chained CBC model. Alternatively, a binarization technique may be applied between all CBC blocks, except the last one, which binarizes the respective possibility vectors.

As shown at step S420c, in the context of the end-to-end training of the model it may be further provided that errors are back-propagated through the chained CBC model to update the model.

As shown at step S430, the method may include a step of investigating the trained model and the classification results. According to an embodiment, this step may include extracting explanations from the trained model by interpreting the intermediated representations (i.e. the outputs of the CBC blocks, except the last one of the chain) as concept probabilities. According to a further embodiment, this step may also include a usage of the trained model to predict the classes of new data samples and interpret the classification process by local explanations (sample-based) and/or global explanations (model-based).

As described above, the present disclosure provides a generic method that learns higher order reasoning by concepts that allow for full traceability of the deep machine learning method. Embodiments of the present disclosure achieve this by one or more of the following aspects (see also FIGS. 3 and 4):
1. Chaining of CBC blocks by interpreting the output of each CBC block, except the last one, as a possibility vector of detected concepts.
2. Ensuring the probabilistic nature of the model by applying a binarization technique between all CBCs blocks, except the last one, or on all primitives after the first block.
3. Stable end-to-end training with improved comprehensibility of provided explanations by only binarizing the primitives.

Embodiments of the present disclosure as disclosed herein provide a crucial step towards high performing yet interpretable AI models. This is important whenever it can be required to be able to assess whether an AI system is compliant with certain regulations or provisions. In this context, embodiments of the present disclosure will be widely applicable. In a general sense, embodiments of the present disclosure provide an important model class that can be used in any application where it is important to build user trust in model predictions, as the model is fully interpretable and therefore explainable by design. This can for example find important applications as an AI augmented human decision support system in areas such as public safety or public services, to name just a few examples.

The architecture proposed herein is generally designed for classification problems. It should be noted, however, that although the model is interpretable by design via a connected probability tree diagram, this does not guarantee that the explanations that can be created by visualizing the learned parameters are easily human understandable. It just means that the parameters and the associated explanations reflect the true reasoning process of the model. To ensure easily human understandable explanations the model might be further constraint or regularized.

Hereinafter, some exemplary use cases for application of a method according to embodiments disclosed herein, will be described in detail.

### 1. Fully Transparent Disease Classification

A first exemplary use case is selected from the health sector and relates to a monitoring of patients and automatically detecting a patient's disease.

According to this use case, a model of chained CBC blocks may be established and trained on available data, as described herein. The available data may include patient related health data obtained from several data sources. For instance, the training data of may include patient data like blood pressure, temperature, EEG, or the like. Alternatively or additionally, the training data may include image data, such as x-rays or ultrasound images.

The model may be trained to output (any kind of) classified disease(s), wherein multiple hot classes are possible. After the model is trained, each parameter and intermediate representation in the network is fully interpretable and can be verified by experts before the model is deployed, as well as, optionally, during deployment.

According to an embodiment, the input for the chained model of CBC blocks may come from a patient monitored in real-time, and the output (classified disease) may include properly prepared visualizations of the intermediate decision process of the network, which may be given to the doctor/physician, i.e., the (medical) expert. Instead of only considering the predicted disease(s), the expert may interact with the system by investigating the visualizations in order to understand which input features are in favour of the predicted disease(s) and which features contradict the predicted disease(s). In particular, the expert is also able to understand which features support the prediction of another disease and how likely is this disease. By this, the expert can verify the prediction before a prescription is prescribed and can be aware of side-effects if the prediction of another disease was likely as well (i.e., when some features or feature combinations supported the prediction of another disease).

### 2. Knowledge Discovery for Disease Classification by Analysing Markers of Blood Tests

A second exemplary use case is likewise selected from the health sector and relates to an identification of unknown indicators and their relations for certain diseases.

According to this use case, a model of chained CBC blocks may be established and trained on available data, as described herein. The available data may include blood test results (markers) of patients including diagnosed diseases.

The model may be trained to output a disease. After the model is trained, each parameter and intermediate representations in the network are fully interpretable and can be analysed by experts. This allows to identify “hidden” patterns that indicate a certain disease. Given a certain disease, correlations between different blood markers can be identified.

According to an embodiment, the network, once trained and verified by experts (by inspecting the intermediate representations, may be used to generate knowledge about unknown correlations between blood markers and diseases. This knowledge can be used to design more efficient blood test machines by, for instance, deploying the verified network to a blood test machine so that, when a blood sample is analysed for a certain disease (input may be coming from an expert, e.g., by suspecting a certain disease), based on the knowledge stored in the network, the machine may automatically select the blood markers to be analysed on the sample in order to confirm or reject the disease. According to an embodiment, this can be realized in a continual/active learning setting. By this automatic (minimal) blood marker selection, the machine is able to perform the disease verification faster than the traditional method of generating a more exhaustive blood test. Additionally, by using the generated knowledge, it is also possible to directly select markers that falsify diseases that are indicated by similar markers (to avoid an incorrect diagnosis). Instead of only returning the blood test results, the machine may be configured to return also a probability for the suspected disease, probabilities of other (similar) diseases, and the explanation for each disease (i.e., why the analysed features triggered the prediction).

### 3. Policy Recommendation for Nature Loss Prevention

A third exemplary use case is selected from the technical field of achieving carbon neutrality and relates to an identification of unknown indicators and their relations for certain nature losses.

According to this use case, a model of chained CBC blocks may be established and trained on available data, as described herein. The available data may include, for instance, satellite image data of the earth or of certain regions.

The model may be trained to output correlations between certain indicators and their relations for certain nature losses. After the model is trained, each parameter and intermediate representation in the network is fully interpretable and can be analysed by experts. The intermediate representations may indicate which past policies or measures were responsible for nature loss (e.g., extinction or decline of certain animal and/or plant species). For example, it can highlight that a growing suburban area led to the loss of healthy trees in a nearby forest. Accordingly, the model may be trained to output a recommendation on what policy/measure to implement for an area of interest, such as how to ensure that a forest does not deteriorate.

According to an embodiment, the chained CBC network may be configured to output (directly) actionable information to either a computer systems or a human operator. For example, it might recommend a certain water system to reroute water from different areas.

As will be appreciated by those skilled in the art, further application scenario can be envisioned in a variety of different technological fields.

Many modifications and other embodiments of the disclosure set forth herein will come to mind to the one skilled in the art to which the disclosure pertains having the benefit of the teachings presented in the foregoing description and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A computer-implemented method for creating an interpretable machine learning classification model, the method being implemented in one or more processors connected to a memory, the method comprising:
defining the model by setting up a chained classification-by-components (CBC) architecture including a number of chained CBC blocks;
end-to-end training of the model on available input data including forward-propagating samples of input data through the chained CBC architecture; and
interpreting an output of each CBC block of the chained CBC architecture, except a last CBC block, as a possibility vector of concepts detected in a respective one of the CBC blocks.

2: The method according to claim 1, wherein each of the CBC blocks includes:
a first stage, in which detection probability measures are determined for each input based on an analysis of the respective inputs for matches with a given set of primitives, and
a second stage, in which probabilities of detected primitives of the set of primitives are analysed to reason about present concepts in the each input.

3: The method according to claim 1, wherein a probabilistic nature of the chained CBC architecture is ensured by applying a binarization technique in between subsequent CBC blocks.

4: The method according to claim 1, wherein a probabilistic nature of the chained CBC architecture is ensured by applying a binarization technique on all detected primitives after the first CBC block of the chained CBC network.

5: The method according to claim 3, wherein the binarization technique applies a Gumble softmax concept or the Implicit Maximum Likelihood Estimation (I-MLE) framework.

6: The method according to claim 1, further comprising:
updating the model based on a back-propagation of classification errors.

7: The method according to claim 1, further comprising:
using the trained model to predict classes of new data samples and to interpret the classification process by sample-based local explanations and/or model-based global explanations.

8: The method according to claim 1, further comprising:
training the model on health related patient data, including at least one of blood pressure, temperature and EEG, and/or image data from patients; and
using the trained model to predict one or more classified diseases of a patient and to obtain explanations of an intermediate decision process of the model for verification by a medical expert.

9: The method according to claim 1, further comprising:
training the model on blood markers from blood test results of patients including diagnosed diseases; and
inspecting intermediate representations of the model for generating knowledge about unknown correlations between blood markers and diseases.

10: The method according to claim 1, further comprising:
training the model on satellite image data of the earth; and
using the trained model to predict unknown indicators and corresponding relations for certain nature losses and to obtain knowledge from intermediate representations of the model regarding which passed policies and/or measures were responsible for a given nature loss.

11: A system for creating an interpretable machine learning classification model, the system comprising one or more processors that, alone or in combination, are configured to provide for the execution of the following steps:
defining the model by setting up a chained classification-by-components, CBC, architecture including a number of chained CBC blocks;
end-to-end training of the model on available input data including forward-propagating samples of input data through the chained CBC architecture; and
interpreting an output of each CBC block of the chained CBC architecture, except a last one, as a possibility vector of concepts detected in a respective one of the CBC blocks.

12: The system according to claim 11, wherein each of the CBC blocks includes
a first stage, configured to determine, for each input, detection probability measures based on an analysis of the respective inputs for matches with a given set of primitives; and
a second stage, configured to analyse the probabilities of detected primitives of the set of primitives to reason about a class of the each input.

13: The system according to claim 11, further comprising binary gates implemented between subsequent CBC blocks of the chained CBC architecture and configured for binarization of the possibility vectors.

14: The system according to claim 11, further comprising binary gates implemented within each CBC block except a first CBC block of the chained CBC architecture and configured for binarization of the primitives.

15: A tangible, non-transitory computer-readable medium having instructions thereon which, upon being executed by one or more processors, alone or in combination, provide for execution of a method for creating an interpretable machine learning classification model, the method comprising:
defining the model by setting up a chained classification-by-components (CBC) architecture including a number of chained CBC blocks;
end-to-end training of the model on available input data including forward-propagating samples of input data through the chained CBC architecture; and
interpreting an output of each CBC block of the chained CBC architecture, except a last CBC block, as a possibility vector of concepts detected in a respective one of the CBC blocks.
